(19) United States
(12) Patent Application Publication
(10) Pub. No.: US 2022/0189486 A1
(43) Pub. Date: Jun. 16, 2022

(54) METHOD OF LABELING AND AUTOMATING INFORMATION ASSOCIATIONS FOR CLINICAL APPLICATIONS

(71) Applicant: AUGMEDIX OPERATING CORPORATION, San Francisco, CA (US)

(72) Inventor: Wen-wai YIM, San Francisco, CA (US)

(73) Assignee: AUGMEDIX OPERATING CORPORATION, San Francisco, CA (US)

(21) Appl. No.: 17/649,648

(22) Filed: Feb. 1, 2022

Related U.S. Application Data
(63) Continuation of application No. PCT/US2020/045634, filed on Aug. 10, 2020.
(60) Provisional application No. 62/884,385, filed on Aug. 8, 2019.

(51) Int. Cl. G10L 15/26; G06N 20/00; G16H 15/00; G16H 10/60

(52) U.S. Cl. CPC G10L 15/26; G16H 10/60; G16H 15/00; G06N 20/00

(57) ABSTRACT

Systems and methods are providing for associating portions of data from a first data file to a second data file. The association may be used to generate machine learning libraries or for other purposes. Exemplary embodiments may include a first data file of a text extraction of a dialog between a clinician and a patient and the second data file are clinical notes obtained from the exchange between the clinician and the patient.

### PRIORITY

This application is a continuation of PCT/US2020/045634 filed Aug. 10, 2020, which claims priority to U.S. Application No. 62/884,385, filed Aug. 8, 2019, which are incorporated by reference in their entirety.

### BACKGROUND

The purpose of annotation, also known as data labelling, is to create a corpus for data analysis or machine learning prediction. This process is useful to build and test effective algorithms to solve complex tasks.

The creation of labelled datasets for clinic visit dialogue to clinical note pairs is relatively unexplored. This is largely due to both the dearth of such data in the past, as well as issues of patient privacy related to protected health information. For example, clinic visit audio are not now nor have they historically been routinely recorded. Clinical notes themselves have only become electronically recorded with the widespread adoption of electronic medical records due to the 2009 HITECH Act as part of the American Recovery and Reinvestment Plan. The issues of patient privacy further limits the amount of data that is accessible to technologists.

Strategies used in other related domains are not immediately adoptable to the clinical environment as the clinical environment and associated data within the clinical environment uniquely combines information extraction, summarization, translation, and natural language generation across two natural language mediums: spoken dialogue and clinical note text. Furthermore, in oppose to dialogue systems, such as credit card or airplane booking transactions, the range of topics and information during a clinic visit is much less narrowly defined.

Even when a corpus is generated for training a machine, transforming dialog language into notes is challenging, especially in the medical context. For example, the spoken dialogue has semantic variations to the written clinical note. The natural dialogue is also riddles with anaphora. In addition, the order of information and organization of data in a clinical note may not match the order of discussion in a clinic visit dialogue. This provides additional challenges in the alignment process. This is illustrated in FIG. 15 in which corresponding information in a matched note and the dialogue sentence appear in the same font style.

Spoken language in clinic visits have vastly different representations than in highly technical clinical note reports. Dialogue may include more frequent use of vernacular and verbal expressions. In contrast, clinical note text is known to use telegraphic, semi-structured language, along with frequent use of technical medical term, medical acronyms and acronyms, which have multiple word senses.

Anaphora is the phenomenon in which information can only be understood in conjunction with references to other expressions. An example of this is the use of pronouns, “John Smith is president of Company A. He is 50 years old this year.” Only by understanding that “he” refers to “John Smith” do we know that “John Smith” is 50 years old. Although there are more complex part-of anaphoric relations, e.g “Liver: There are three tumors in the right lobe. Lesion in Segment 6 is 3.0 cm. The other lesions are 1.0 cm.” Here it is understood that the 3.0 cm lesion is one of the three tumors in the right lobe of the liver (since segment 6 of the liver is in the right lobe). The other lesions numbers to 2 (since there are three total lesions, and one has already been previously described) and are also located in the right lobe. While anaphora occurs in all naturally generated language, in dialogue, it may appear across multiple speaker turns many sentences apart.

Additional difficulties are also encountered in creating machine generated matching of dialog sentences to clinical note sentences because the clinical note content includes filtered and summarized information from the dialog. What information content from the dialogue becomes part of the clinical note is often filtered and summarized. Therefore, not all dialogue information is represented in the clinical note, and information that is represented may be summarized. Therefore, the form, structure, and content between the dialog sentence to the mated clinical note is not constant. In addition, the clinical note content may include sources other than that from the dialog sentence. Not all clinical note content is therefore represented or corresponds to dialogue content. Information may come from a clinical note template, various parts of the electronic medical record, or may be taken from conversations not included in the clinical-patient visit. Therefore, not all clinical note sentences can be aligned to dialogue text content.

### SUMMARY

Exemplary embodiments include system and methods for creating a parallel corpora for machine learning. The relationships may be created between dialog sentences from a dialog exchange with a patient and clinical notes made about from the dialog sentences. In an exemplary embodiment, the dialog sentences are any natural language extraction of information from a patient, such as through patient questioning, or discussion during an examination. The clinical note sentences may be the diagnosis statements and impressions made during and/or after the exchange with the patient. The clinical note sentences include the extracted relevant information from the dialog sentences in clinical language and format.

In an exemplary embodiment, an annotator can match sentences of transcribed dialogue text from a clinic visit to sentences of a corresponding finished clinical note. In an exemplary embodiment, the dialogue transcription is linkable to time-stamped transcriptions, as well as to its original audio media files. The clinical note is likewise linkable to originating structure, (e.g. templated text, drop-down selections, etc.). The matched annotations may be characterized by labels that relate to the type of connection between a dialogue sentence and the clinical note sentence. The relationship between a note sentence and dialogue sentences are one-to-many, and may be hierarchical. The annotations may be stored separately and reference the dialogue and clinical note by text offset location.

Exemplary embodiments of the generated parallel corpora from the sentences and methods described herein may be used for training automatic language generation systems. Exemplary embodiments of systems and methods for training automatic language generation systems including first creating parallel corpora of source dialogue text to target narrative text from comparable corpora, and then using the generated corpora to train classification and generation systems.

In an exemplary embodiment, the source dialogue text is clinical conversation dialogue from a clinician and patient visit. The target narrative text is the clinical note.

The sentence alignment system takes as input a paired source dialogue and a target narrative text document and determines whether each target text sentence can be paired with zero or more sentences from the source dialogue. The identified dialogue sentences are organized into one or more labelled sets according to predefined label schema. Thus, the resulting target document can be associated with one or more sets of sentences from the source dialogue. Exemplary embodiments of this system may be used to create training data that can be used for multiple purposes, including a system for language generation for the target sentences from source dialogue sentences, training data for dialogue sentence organization, or computer-assisted annotation of alignments.

Exemplary embodiments described herein may use a multi-step divided approach for generating sentence alignment. First, sentences from a dialog data set may be identified to be purely structural (such as having no patient information), or are related to a dialogue command related to note structure. These dialog sentences may be marked and removed from further processing. Then, the system and methods may identify dialog sentences to be part of default template values are given a default label reserved for templates. Next, using a learned model, one-to-one high similarity alignments are classified into several one sentence labeled sets. Features may include similarity features, speaker features, and lexical and semantic features. Any number of machine learning algorithms e.g. support-vector machines, decision trees, may be employed in this step. For more complex one-to-many alignments, exemplary embodiments may include creating candidate sets from the dialog sentences using a sliding window for sentences and proximal questions and answer sentences. These sets may be scored with each clinical note sentence based on a feature-based hand-crafted score, optimized from training data.

An exemplary embodiment may include a final module configured to optimize associations with multiple sets that decides inclusion or exclusion of multiple sets and whether sets should be merged or repartitioned. Logic to associate, merge, or repartition decisions may be hand-crafted from training data or may be statistically based. Given the best alignment of sets, a note sentence may be assigned the alignment if a final classifier determines acceptance.

Exemplary embodiments of the systems and methods described herein may include sentence alignment configured to assign each clinical note sentence with 0 or more sentences from the dialogue, where the sentences can be grouped in a labelled set.

### DRAWINGS

FIG. 1 illustrates exemplary data sources that feed into an interface for marking annotation according to embodiments described herein.

FIGS. 1A-1C illustrate exemplary interfaces for marking annotations. These include selecting an item from the source data set, and providing zero or more items from a second data set in which the user may provide associations, comments, tags, or other data items.

FIGS. 2A-2C illustrate exemplary interfaces for providing annotations that may be added.

FIGS. 3A-3C illustrate exemplary interfaces for providing annotations that may be added by selecting sets and adding desired tags according to embodiments described herein.

FIG. 4 illustrates an exemplary visual depiction of an association of information according to embodiments described herein.

FIG. 5 illustrates an example of associations that may be made between portions of difference corpuses according to embodiments described herein.

FIG. 6 illustrates an example visual representation of annotations with accompanying text.

FIG. 7 illustrates an example interface with exemplary explanations of sections of the user interface.

FIG. 8 illustrates example interface for placeholders of information.

FIGS. 9A-9B provides example tables for keeping audio dialogue data, including metadata such as speaker, references of matchable sentences, and/or related original data.

FIGS. 10A-10B illustrate exemplary tables for keeping clinical note data and tracking references of matchable sentences of the original data.

FIG. 10C illustrates an example table for storing associated sources of matching information.

FIGS. 11A-11B provide example tables for keeping stored data.

FIGS. 12A-12B illustrates an exemplary user information for displaying information as described and/or entered into embodiments described herein. The first data set, such as a dialog corpus, may be displayed (right side); and a second data set, such as a clinical note corpus, may be displayed (left side), with sets, tags, annotations, or other data provided therewith (middle side).

FIG. 13 provides examples of how passages can be grouped hierarchically, such as for use with repeated information found in different sentences.

FIG. 14 illustrates an exemplary system input and output for the sentence alignment system.

FIG. 15 illustrates an exemplary associated dialogue (shown in the middle) and clinical note (shown on the left) shown along with their associated paired sentences organized into labelled sets (shown on the right).

FIG. 16 illustrates an example use of a corpus having aligned pairs of sentences within two corpus to be used to create a language generation system which would take in automatic speech generation data and output clinical note sentence suggestions.

FIGS. 17A-17C illustrates an exemplary flow diagram for determining tags to be associated with one or more sentences within one or more corpus according to embodiments described herein.

FIGS. 18A-18B are exemplary module representations according to embodiments described herein.

FIG. 19 illustrates an exemplary system diagram according to embodiments described herein.

### DESCRIPTION

The following detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. It should be understood that the drawings are diagrammatic and schematic representations of exemplary embodiments of the invention, and are not limiting of the present invention nor are they necessarily drawn to scale.

Exemplary embodiments described herein include systems and methods for annotating that may explicitly match sentences of a clinic visit dialogue to sentences in a clinical note. A single note sentence can be matched with a set of sentences from the dialogue transcript. A single note sentence can be matched with zero sets or more than one set of sentences from the dialogue transcript. Exemplary embodiments of the system and method permit an association of one or more tags with a matched set of note and dialog sentences. The matched set of note and dialog sentences may be associated with one of several tags to indicate the mode of information verbalization or type of information transformation, e.g. dictation, statement, question-answer. The note sentence may also be tagged with additional labels such as incomplete or inferred. Per a single note sentence, sets may be related to other sets in a hierarchical manner.

Exemplary embodiments described herein include system and methods for associating, tagging, and/or annotating two different data sets for providing a library for machine learning. The two different data sets may be corpus of text each comprising a plurality of sentences. The two different data sets may be in the same language, but may include different dialects, different terminology, different styles, etc. For example, a first data set may be in a verbal dialog format between two individuals. The first data set may also include more than one different style. For example, the first data set may include dialog exchanged between more than two people, statements made to the system by one person, statements made in observation or in response to actions (instead of verbal exchanges), etc. A second data set may be a conformed data set that extracts information, summarizes, formalizes, and/or consolidates information from the first data set. In an exemplary embodiment, the first data set comprises a text recording of the audible exchange between a patient and clinician (nurse, doctor, practitioner, etc.), while the second data set comprises clinical notes extracted from the exchange between the patient and clinician that generated the text recording. Exemplary embodiments may include dialog sentences related with note sentences from a clinical exchange between a patient and a care giver, or individual having a relationship to the patient and/or care giver.

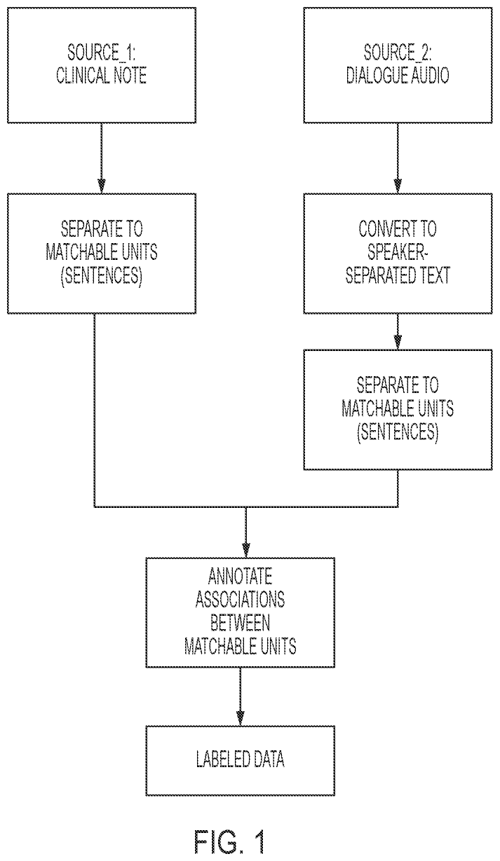

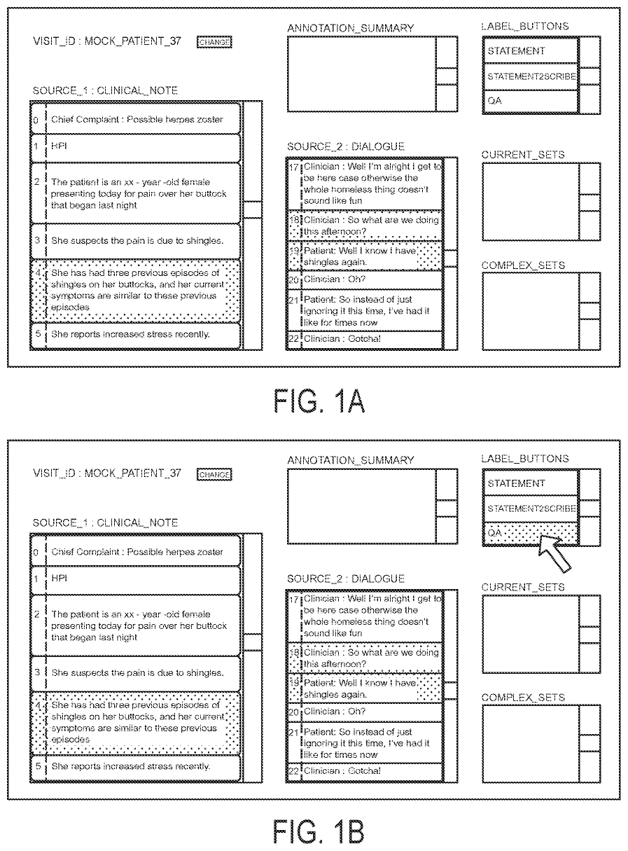

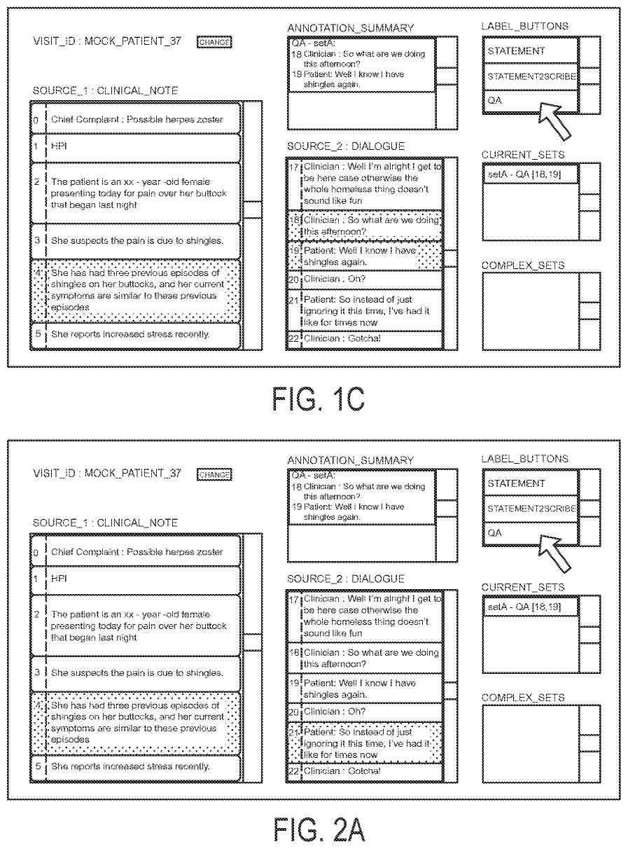

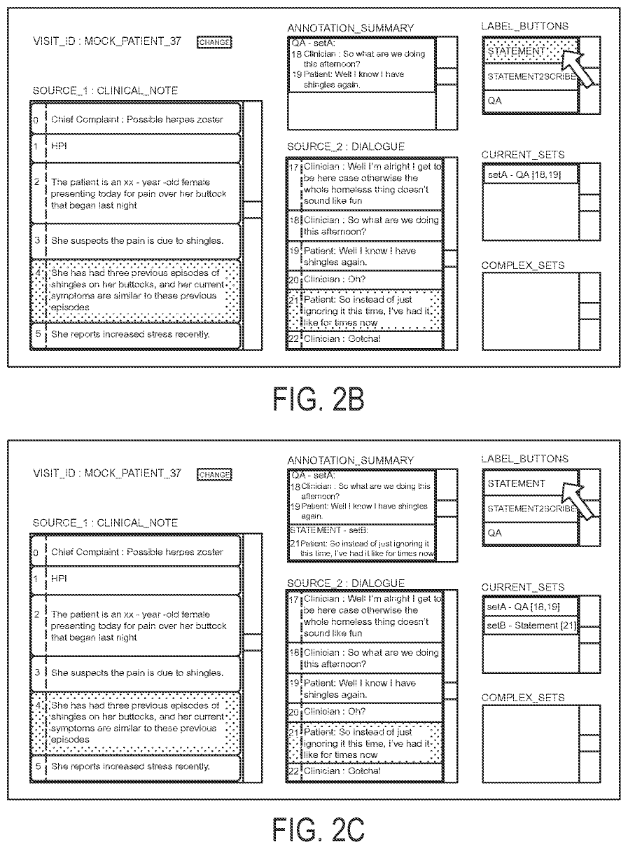

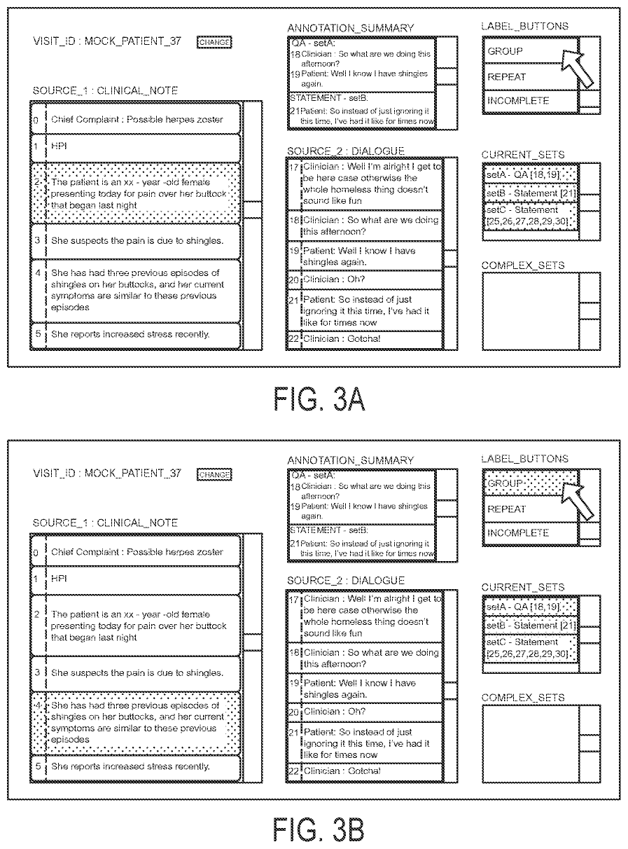

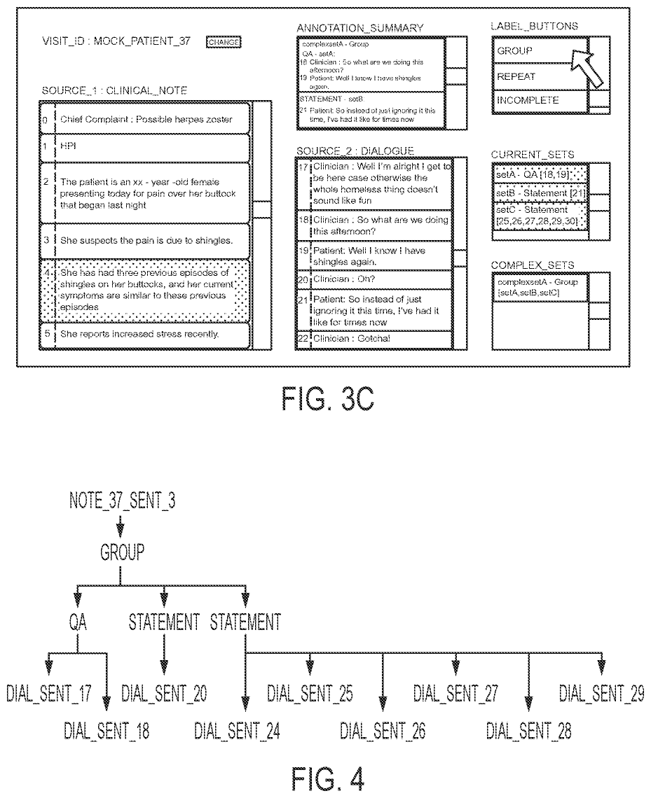

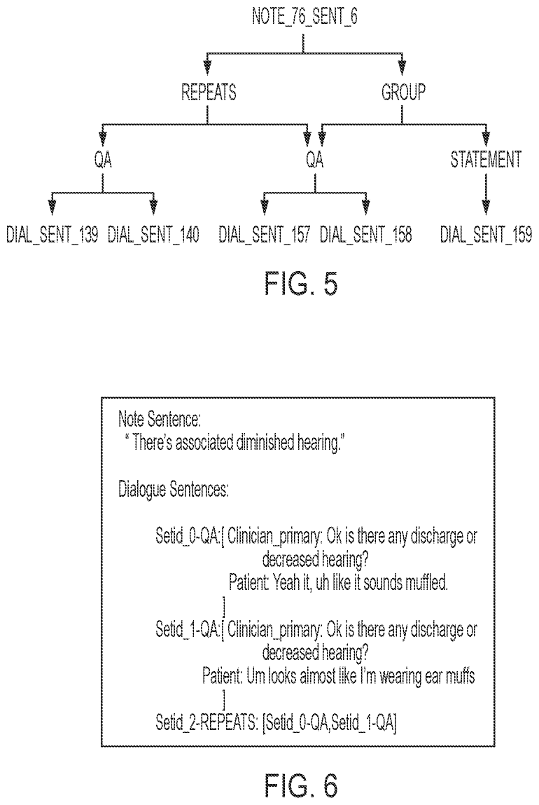

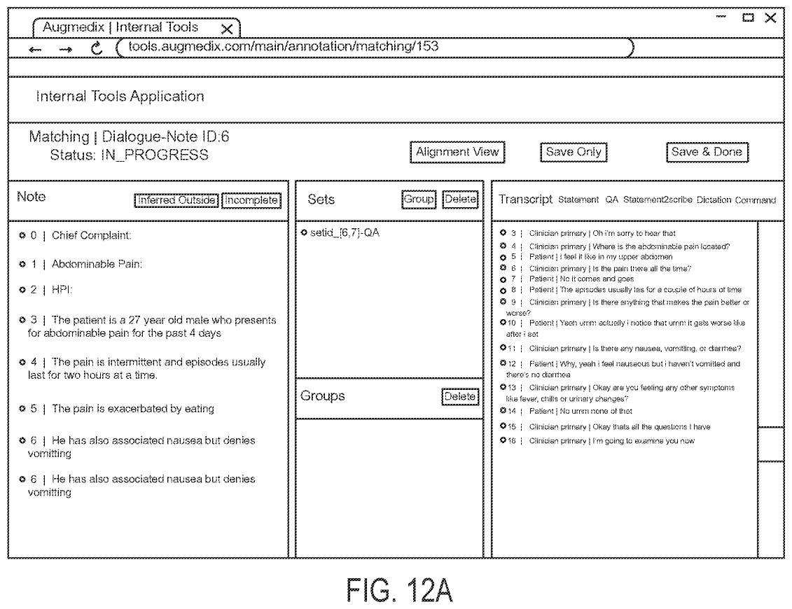

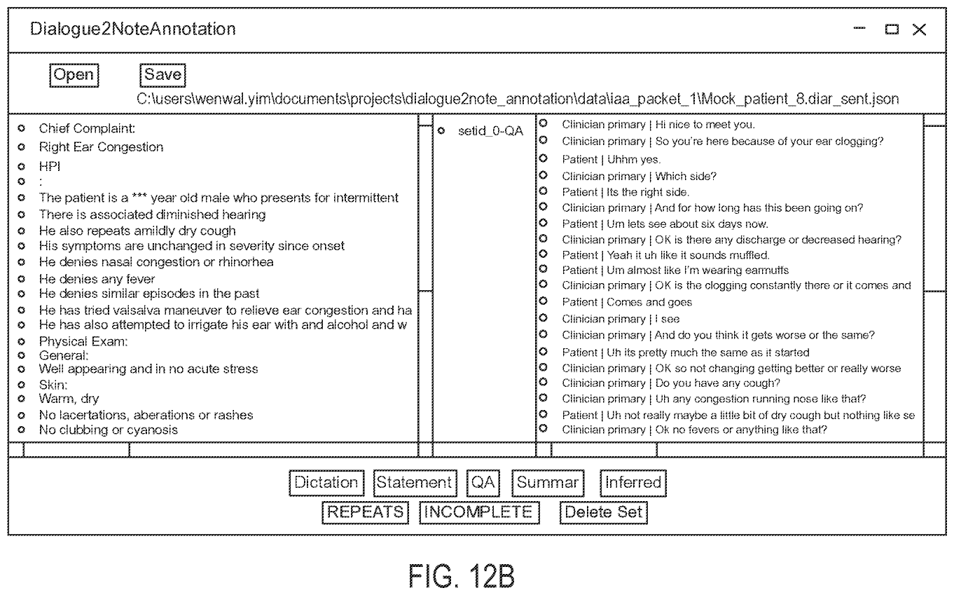

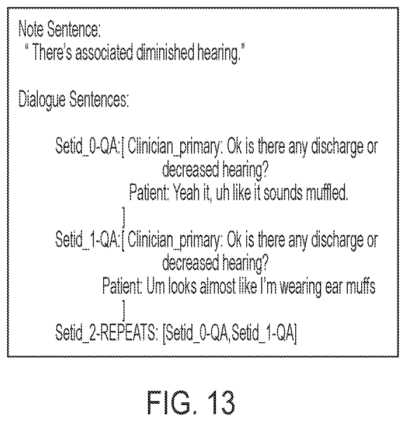

Exemplary embodiments of the system include an interface for receiving input from a user for creating the associations, tagging, and/or annotating dialog and/or note sentences. An example of an interface that records this annotation is shown in FIGS. 1-3, with its associated annotation shown in FIG. 4. FIGS. 5-6 show additional examples of complex annotations. Another example of an interface that records this annotation is shown in FIGS. 12A-12B, with its associated annotation shown in FIG. 13.

Exemplary embodiments of using the system described herein and/or the method associated therewith may result in paired corpuses, from which a technologist can retrieve the source dialogue sentences from which a clinical note sentence was created from, and vice-versa.

In an exemplary method, a system may be configured to display information to a user and receive information from a user. For example, a system may display information from two different source and/or in two different formats. In an exemplary embodiment, the first and second information may be related. For example, the first information may be derived or related to the second information. The displayed information may include a user input for indicating a selection of a first specific information from one of the two different sources and a second specific information from the other of the two or more different sources.

For example, as shown in exemplary FIG. IA, a first displayed information includes clinical notes created from a clinical exchange between a patient and a practitioner. The clinical notes may be derived from a direct exchange with the patient, such as through a dialog with or examination of the patient. The clinical notes may be derived from an indirect exchange such as information received from sources other than the patient, or from the patient indirectly, such as through questionnaires, written inquiries, etc. As shown in exemplary FIG. IA, a second displayed information includes information that is the source of the first displayed information. For example, the second displayed information may be a full or partial transcript of a dialog with the patient. The second displayed information may also or alternatively be information received from a questionnaire, written inquiry or other source.

As shown in exemplary FIG. IA, the system may also display options for user input. The user input may be a system and method for linking or associating content from the first displayed information and the second displayed information. As illustrated, each sentence (data segment such as a line) of the first displayed information and each sentence of the second displayed information can be selected. For example, the user may select a button corresponding to a first specific information from the first displayed information and a second specific information from the second displayed information and indicate an intention to link the selected first and second specific information. The system may also receive additional information regarding either or both of the first specific information and/or the second specific information and/or the relationship between the first and second specific information. For example, as seen in FIG. IA, the system may display one or more tags that a user may select when also selecting specific information from a first corpus or linking specific information to another specific information of a second corpus.

In an exemplary embodiment, the tags may be used to assist in data analysis and machine learning. In an exemplary embodiment, the tags may be related to or identify a form of information, a source of information, a relation between information, and combinations thereof. For example, tags may indicate the form of information, such as, for example, whether an identified dialog is in a question answer form or as a statement. The tag may indicate the source of information (which may or may not be related to the form of information). For example, the tag may indicate whether the information is from a dictation of the practitioner or other observer of the patient, or whether it is a question and answer form. The tag may indicate a relation between the first specific information and the second specific information between different information sets. In the specific example of clinical notes and captured dialog, the tag may indicate whether the clinical note is an inference from the dialog or a summary of the dialog or directly related or obtained from the dialog such as a dictation. The tag may indicate a relation between a second specific information and a third specific information from the same information source as it relates to the first specific information of a different information source. For example, when a first and second specific information are related between two different information sources (i.e. the clinical notes and the transcription of a patient dialog) and a first and third specific information are related where the third specific information is from the same (or different) information source than the second specific information, the second and third specific information is linked through the first specific information (i.e. the clinical note). A tag may be used to indicate how the second and third specific information pieces are related, such as, for example, whether the information is a repeat of each other. Other tags may also be used, such as to indicate whether information is incomplete, whether information is in passive or active voice, or other identifier of the information itself or relation to the information or link between information.

Exemplary System

In an exemplary method, a system may be configured to display information to a user and receive information from a user. For example, a system may display information from two different source and/or in two different formats. In an exemplary embodiment, the first and second information may be related. For example, the first information may be derived or related to the second information. The displayed information may include a user input for indicating a selection of a first specific information from one of the two different sources and a second specific information from the other of the two or more different sources.

In an exemplary embodiment, the first information and second information may be corpus of text from the same language in different formats. The first format may be an extraction of information from the first format. For example, the first format may include summary, analysis, extraction, consolidation, and combinations of the information from the second format. The first format may be a format for unifying the presentation of information, while the second format is creates a raw data set for extraction into the first data set. The first corpus may be different from the second corpus in the terminology used to describe the same thing (whether an object, sensation, symptom, action, experience, etc.). The first corpus may be different from the second corpus in the size of the related information. For example, the second corpus may be expressed in more works, over multiple sentences, etc., or may be duplicative in the presentation of the same information, while the first corpus represents data in a concise (few words), in a single sentence, non-duplicative, and/or uniform terminology.

For example, as shown in exemplary FIG. 12A, a first displayed information includes clinical notes created from a clinical exchange between a patient and a practitioner. The clinical notes may be derived from a direct exchange with the patient, such as through a dialog with or examination of the patient. The clinical notes may be derived from an indirect exchange such information received from sources other than the patient, or from the patient indirectly, such as through questionnaires, written inquiries, etc. As shown in exemplary FIG. 12A, a second displayed information includes information that is the source of the first displayed information. For example, the second displayed information may be a full or partial transcript of a dialog with the patient. The second displayed information may also or alternatively be information received from a questionnaire, written inquiry or other source.

As shown in exemplary FIG. 12A, the system may also display options for user input. The user input may be a system and method for linking or associating content from the first displayed information and the second displayed information. As illustrated, each sentence (represented as a line) of the first displayed information and each sentence (represented as a line) of the second displayed information include radio buttons adjacent specific information. The user may select a radio button corresponding to a first specific information from the first displayed information and a second specific information from the second displayed information and indicate an intention to link the selected first and second specific information.

The system may also receive additional information regarding either or both of the first specific information and/or the second specific information and/or the relationship between the first and second specific information. For example, as seen in FIG. 12A, the system may display one or more tags that a user may select when also selecting specific information or linking specific information to another specific information. The tags may be used to assist in data analysis and machine learning. In an exemplary embodiment, the tags may be related to or identify a form of information, a source of information, a relation between information, and combinations thereof. For example, tags may indicate the form of information, such as, for example, whether an identified dialog is in a question answer form or as a statement. The tag may indicate the source of information (which may or may not be related to the form of information). For example, the tag may indicate whether the information is from a dictation of the practitioner or other observer of the patient, what it is a question and answer form. The tag may indicate a relation between the first specific information and the second specific information between different information sets. In the specific example of clinical notes and captured dialog, the tag may indicate whether the clinical note is an inference from the dialog or a summary of the dialog or directly related or obtained from the dialog, such as a dictation. The tag may indicate a relation between a second specific information and a third specific information from the same information source as it relates to the first specific information of a different information source. For example, when a first and second specific information are related between two different information sources (i.e. the clinical notes and the transcription of a patient dialog) and a first and third specific information are related where the third specific information is from the same (or different) information source than the second specific information, the second and third specific information is linked through the first specific information (i.e. the clinical note). A tag may be used to indicate how the second and third specific information pieces are related, such as, for example, whether the information is a repeat of each other. Other tags may also be used, such as to indicate whether information is incomplete, whether information is in passive or active voice, or other identifier of the information itself or relation to the information or link between information.

Exemplary Method

The system according to embodiments described herein may be used in a method for identifying and storing information. Exemplary system and methods described herein may be used to generate one or more datasets for which machine learning algorithms may be applied. Exemplary embodiments may be used to generate machine learning systems and methods for relating and generating clinical notes from clinical data, including, but not limited to, dialog with a patient, questionnaire responses by a patient, received sensor information, patient provided information, and/or clinician provided information. In an exemplary embodiment, the machine learning algorithm may recognize preferences of one or more users to generate the desired report or information.

In an exemplary method for building a dataset, the first step may include displaying information to a user. The method may also include receiving and/or generating the information prior to displaying it to a user. The information may originate from one or more sources. In an exemplary embodiment, the information includes at least two sources of information that are simultaneously displayed to a user. The method may include generating the second information from a first information to supply the at least two sources of information. In an exemplary embodiment, the second information may be conclusions, notes, impressions, summary, or other relationship to the first information that may include raw data. In an exemplary embodiment, the first information includes information received through dialog with a patient, and the second information are clinical notes based on the first information.

In an exemplary method for building a dataset, the second step may be to receive user input for selecting portions of the displayed information. The receiving user input may include receiving multiple user inputs, at least one from each of the two or more sources of information. For example, the method may include receiving a first user input selecting a first specific information from a first source of information and receiving a second user input selecting a second specific information from a second source of information different from the first source of information. The method may include creating a link between the selected first specific information and the selected second specific information.

In an exemplary method for building a dataset, the third step may be to receive additional information about the dataset. For example, additional information may relate to any of the displayed information, selected first specific information, the selected second specific information, the link between the selected first specific information and the selected second specific information, and combinations thereof. In an exemplary embodiment, the received additional information is through a user input. In an exemplary embodiment, the received additional information is defined as a tag (category) to characterize, identify, or relate to the information.

In an exemplary method for building a dataset, the fourth step may be to store data associated with one or more steps of the method into a database. In an exemplary embodiment, the system may include create a unique identifier for an entry into the database. The method may include storing information of or relating to a first specific information from the first information source, the second specific information from the second information source, the tag, the unique identifier, and combinations thereof. The stored information may include an entry for a unique combination of first specific information, second specific information, and a tag. The stored information and individual entries of the stored information may include any combination of one or more specific information from the first source, one or more specific information from the second source, zero or more tags, user identity of any data piece within the entry, source identity of any data piece within the entry, time or creation information of any data piece within the entry, relationships or pointers to any other entry or other data piece within the dataset, etc.

In an exemplary method for building and using a dataset, the fifth step may include displaying stored information to a user. The step of display may also include receiving another user input from a user indicating a desired display format and displaying the stored information according to the desired display format.

In an exemplary embodiment for building a dataset, one or more steps of the method may be repeated. For example, the method may include receiving a third and/or fourth user input selecting third specific information related to the first information source and a fourth specific information related to the second information source and one or more tags or additional information and storing within the database. As another example, the method may include receiving a fifth user input selecting a fifth specific information related to the first information source. The fifth specific information may be linked to the second specific information of the second specific information defining a single to multiple linked between the second specific information from the second information source and the first and fifth specific information from the first information source.

Exemplary systems and methods may be used to define data sets in which an entry of the data set may directly associate a first data piece to a second data piece with additional information. The first data piece may be from a first data source and the second data piece may be from a second data source. The second data source may be generated from the first data source. The first data source may include raw data. In a specific example, the first data source is a verbal dialog between two or more individuals, and the second data source is a summary, notes, impressions, from the verbal dialog. For example, the verbal dialog may be from a patient-practitioner exchange, and the second data source may be the clinical notes generated from the patient-practitioner exchange. The systems and methods may provide a direct correlation between the raw or source data from the first data set to the generated data of the second data set. The system and methods may provide additional data to also be associated with the different data and/or in the link between the data sets.

Exemplary embodiments may include any combination of tags to facilitate or achieve the objectives described herein. Exemplary tags may include those provided below and described in Tables 1-3, or otherwise described herein in any combination.

TABLE 1
Tagset when clinician addresses the scribe

| | | # Dialogue |
|---|---|---|
| Tag | Description | Lines |
| Dictation | Clinician spoken information (to scribe) | Nonzero, |
| | that is intended to be dictated | maximum |
| | | continuous |
| Command | Used for giving directions regarding the | Nonzero, |
| | note, e.g. “change to medicare | maximum |
| | template” | continuous |
| Statement2Scribe | Non-dictation, non-command statements | Nonzero, |
| | addressing the scribe | maximum |
| | | continuous |

TABLE 2
Tagset for normal visit duration

| | | #Dialogue |
|---|---|---|
| Tag | Description | Lines |
| Statement | Spoken utterance by patient or clinician, which | Nonzero, |
| | is mentioned in note almost identically with | maximum |
| | minor variations | continuous, |
| | | same speaker |
| QA | Captures question answer form information. | 2 question/ |
| | | answer or a |
| | | sentence/ |
| | | confirmation |
| | | response |
| | | 1 question |
| | | OK as well |
| Inferred- | Used for cases with templates -- Captures | 0 |
| Outside | cases in which information in the note is not | |
| | mentioned in the dialogue at all. | |

TABLE 3
Tagset for higher level tags

| Tag | Description | #Sets |
|---|---|---|
| INCOMPLETE | Extra tag for a note sentence if there are | 0 |
| | parts of it not mentioned in dialogue | |
| GROUP | Used to group sets, for purposes of | Nonzero |
| | hierarchical set relations | |

The exemplary tag, “Command”, demarks portions of the dialogue where the provider is issuing a command related to how the note content should be organized or changed. These tags should be the only ones that are attached to a note section OR a template-place holder. For example, the clinician may use specific language to identify a direct command to the system or annotator. For example, the clinician may state “for PE, use normal male template” or “please insert the risks and benefits template for tonsillectomy”. The keyword “template” may be used to trigger the identification of a command and corresponding instruction set.

The exemplary tag “Dictation” may be used to demark portions of the dialogue which may include directions to the system to create a note directly from the dialog text. For example, the clinical note is essentially a cut and paste or close approximation of the language used from the dialog text to the client note. The clinical note text may encompass fully all the dictation text or vise versa. A dictation tag is used based on the assessment of the relationship between the sentences between each corpus. The following differences may not count against differences or affect whether the dictation tag is applied between the corpuses: punctuation differences; prior or ending extra segments; repeated words; disfluencies (such as “um” or “uh”); articles (such as “a”, “the”, “then”); language that denotes speech additions (such as “and then”, “also”, “also add”); language that denotes punctuations (such as “period”); language that denotes numbers (such as “one”, “two”, “number four”); and combinations thereof. Exemplary differences that are considered for determining whether sentences between corpuses use the “dictation” tag include whether common words are re-orders, if matching number of words does not include a medical term (such as a disease name and/or body part) and/or does not include an adjective or verb. An exemplary algorithm for determining whether a dictation tag should be used includes trying to find matching locations, identifying anything the counts and/or does not count toward the assessment.

The exemplary tag “Statement2Scribe” may be used to categorize information that comes from statements in the dialogue but not directed at the scribe, and therefore not meant for direct dictation. For example, a clinician may make a conclusion or statement about the diagnosis, such as, a dialog statement of “left tympanic membrane is normal” may result in a clinical note of “left TM is normal”.

The exemplary tag “inferred-outside” can be used to refer to a note sentence coming from a template item. For example, this tag may be used to track which patient information is actually checked during the visit, versus what is pre-written. An exemplary note may include “Extremities: no edema or obvious deformity. Full ROM in all joints.” may not have a corresponding dialogue text and may be marked with the inferred-outside as it does not include pre-filled information from a template.

The exemplary tag “statement” may be used for clinician to/from patient dialog that comes from statements in the dialogue, but not directed at the scribe or meant for direct dictation. For example, a patient may state “yea and y medications, I need refills” which can be correlated to the clinical note “requesting refills for medications”,

Another exemplary tag for use between clinician/patient exchanges may include a tag “QA” to denote a question/answer pair. This may include statements by the clinician or patient that require or may have a confirmation from the other person in the dialog. For example, the dialog may include a question answer pair of “bowel habits ad bladder habits ok? Yea” that gets correlated to a clinical note of “he denies any gastrointestinal or genitourinary problems.” Another example may include a dialog pair of “I have here that you are on Cymbalta. Yes” that relates to clinical note “Currently taking Cymbalta for nerve pain.”

An exemplary tag “incomplete” may be used in addition to other tags. It may be used to indicate whether or not there is information in the note sentence that is not accounted for with other annotations. For example, a response may not appear within a captured text of a dialog exchange, such as a question in the dialog “any recent travel?” that does not have an answer, but there is a clinical note that states “denies recent travel.” Exemplary identification elements may be used to indicate whether this tag can be used. For example, the incomplete tag may be used if in the clinical note line one or more of the following elements cannot be found in a corresponding dialogue line, but other elements can be found: medical terms (such as symptoms, diseases, treatments, tests); anatomic locations (including directional indicators such as laterally, left, right, bilateral); quantities (such as dosages, frequencies), aggravating and/or alleviating factors; temporal aspects (such as times, dates, durations, time frames); lifestyle actions or events (such as exercise, adoptions or changes, living with family, drinking); experiencer (if not the patient); and combinations thereof.

An exemplary tag “group” may be used for dialogue information that is collated and re-expressed in the clinical note as a summary that links the dialogue sets together. This may occur or be indicated by a dropped subject, use of pronoun, references, or use of determiner references (such as “this” or “that). The “group” tag may also be used when information for which additional logical reasoning or deduction is required. For example, the patient may be relaying a trip that is planned, in which the clinician interjects a question, and the patient, answers and completes the idea from the original statement. The patient may state that “I′m going to England on the thirteenth”, which the clinician interjects “of this month”, and the patient states, “gone for two weeks”, and the clinical note captures “scheduled for a trip to England on the thirteenth for two weeks.” Logical reasoning may occur over multiple sentences that can include the group tag to indicate the combination of these sentences. For example, an exchange with a clinician may start with a question, such as “so you had this for a long time?” with further dialog, “yeah”; “and you are hold old now?” “twenty six”; “when you were like in high school uh did you get tonsil infections?” “Yes” in which the clinical note may be “symptoms have been present for several years but have worsened over the past 6 months.”

In an exemplary embodiment, the “group” tag is used when an explicit subject is not provided in the dialog and in the information currently provided in the dialog. This may occur when a conversation evolves, and the subject is replaced with a pronoun, a determiner, or is dropped entirely. The dialog sentence that provides the subject from earlier in the conversation can be indicated with the later dialog information with the “group” tag.

Exemplary embodiments may provide corpus relationships within different sections. For example, the related data sets within a corpus may be designated within a specific visit stage. Table 4 provides an exemplary guide for which information from the dialogue can be considered for marking text in the note under certain sections. For example, certain parts of a clinical note may have implied intake procedures. “History of Present Illness” and “Review of Symptoms” may be when clinicians verbally interview their patients, while “Physical Examination” may be used when the clinician will physically examine and obtain independent and/or direct information from the patient.

TABLE 4
Visit stages for note sections

| Visit Stage | Sections |
|---|---|
| Discovering reason for | Chief Complaint (CC) |
| visit/Verbal examination | History of Present Illness (HPI) |
| | Interval History |
| | Review of System (ROS) |
| | Social History (SHx) |
| | Past Medical History (PMHx) |
| | Routine Health Maintenance (RHM) |
| Physical examination | Physical Exam |
| Detailing treatment or | Assessment and Plan |
| further investigation | Impression |

In an exemplary embodiment, portions of the dialog may be prioritize or chosen to be related with a clinical note over other portions of the dialog. To make annotations tractable and reproducible, high level annotation rules may be implemented. For example, for a given note sentence, the parts of the dialogue that gives the most detailed account of the information contained in the clinical note is the one used to associate with the clinical note. The identified dialog sentence(s) can be used in the association while parts of the dialogue that provide “part of” or “piecemeal” accounts can be ignored or identified as a lower priority source of information.

In certain instances, information in the clinical note will come from outside information not included in the dialogue. For example, laboratory values can be inputted automatically from electronic medical record data. In these instances, those parts of the note are left unmarked. Therefore, information, sentences, or other data segment within a clinical note may not have an associated correlation to information, text, sentences, or other data segment within the dialog text if: a section header in a template (and not involved in a COMMAND or other patient-specific information is in the line), and/or nothing from the clinic visit conversation can generate the note sentence.

Exemplary Beneficial Use Cases

For one skilled in the art, the benefits of this annotation may be many-fold. Annotations can be used to build classifiers for dialogue sentence relevancy classification. For example, for incoming dialogue, should the sentence be ignored or not. This can be further specialized for data collected from particular specialty or per specific note types. Furthermore, a classifier can be built to automatically group related relevant sentences. For paired dialogue and clinical notes, an automatic matching algorithm may be used to create a paired corpus of matched associations. This can serve as pre-annotation for a second human review, or be a form of noisily generated corpora. The matched associations corpus can then be leveraged for natural language generation and data analysis tasks. In the context of natural language generation, the matched text may be conducive for sequence to sequence modelling, information retrieval based natural language generation methods as well as information extraction and template-based generation methods. For data analysis, this data can be used to analyse the linguistic similarities of matched text, as well as analyse the percentage of medical terms, question-answer, and small-talk in dialogue. This can help characterize individual providers, specialties, or organizations for business purposes as well as for machine learning meta-data needs.

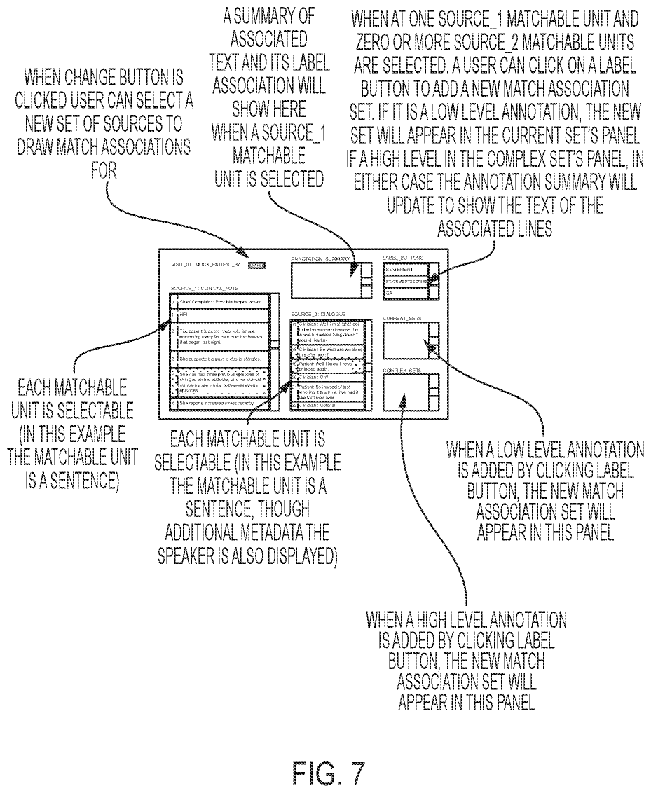

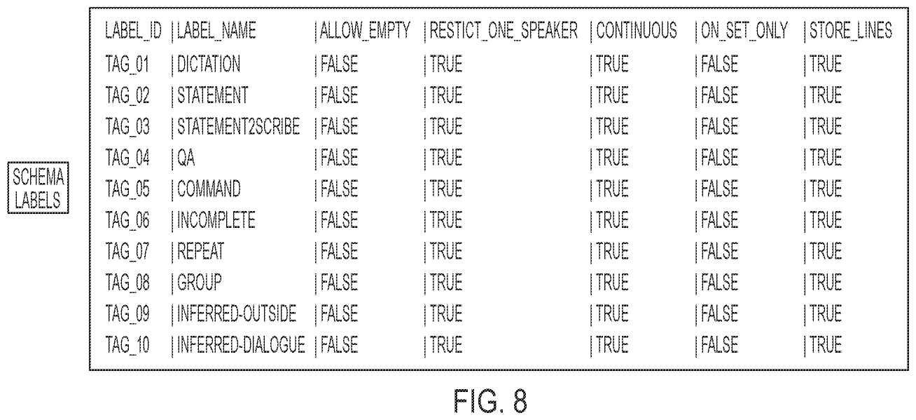

FIGS. 7-12B illustrate exemplary interfaces according to embodiments described herein.

An exemplary embodiment of the system and methods described herein may include audio processing of an exchange between two or more people. During a clinic visit, audio and/or visual data streams may be saved and may be processed such that the parts of the audio related to a speaker can be identified and marked by time stamped. Each segment can further be divided by sentence before given to the user to annotate. For an example, the method may use a transcription step for which the audio is transcribed into text. These steps may be done by a human annotator however speech-to-text services may serve as alternative or augmenting tools. Along with the final dialogue data, the time-stamped offsets per sentence would be stored with references to the origin data.

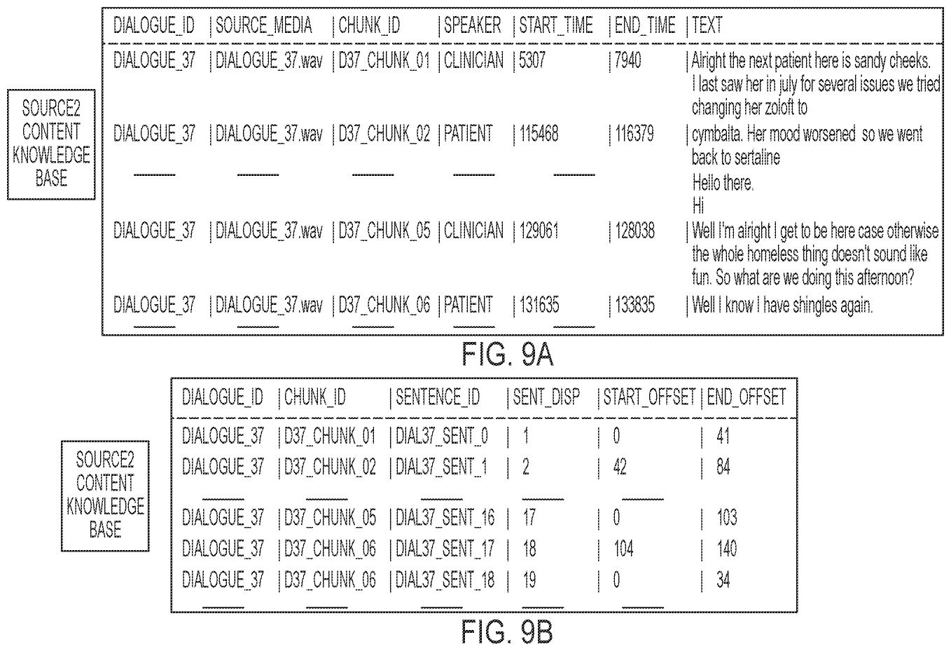

FIGS. 9A-9B illustrate example tables keeping audio dialogue data, including metadata such as the speaker, and references of matchable units (e.g. sentences) related to the original data.

An exemplary embodiment of the systems and methods described herein may include processing of the first data set. This clinical note processing can include representing the clinical note as free text that can be automatically sentence tokenized using a tokenization model. The final sentence data in the tokenized form can be stored with references to the original data.

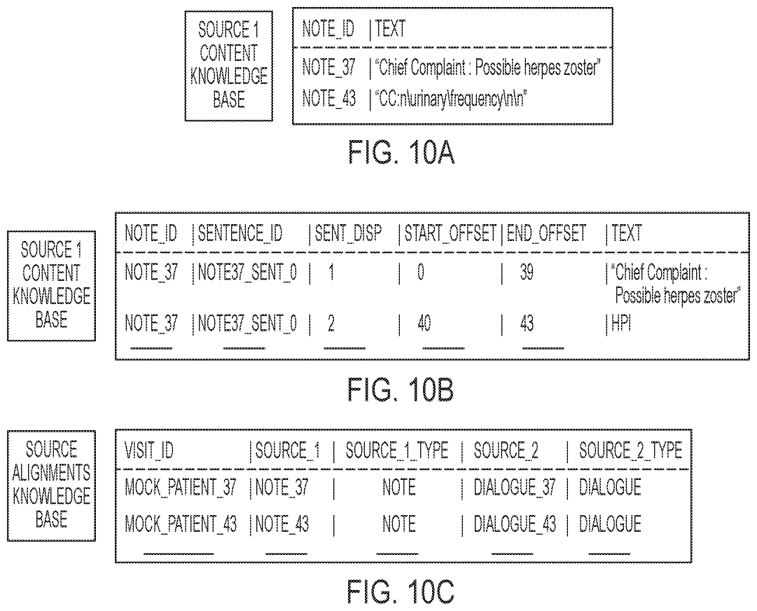

FIGS. 10A-10B illustrate exemplary tables keeping clinical note data and keeping references of matchable units (e.g. sentences) related to the original data.

An exemplary embodiment of the systems and methods described herein may include data retrieval for serving and displaying to a user in a user interface. A database can hold references to which clinical note and dialogues pertain to the same clinic visit. The retrieval method can be able to pull the sentence tokenized dialogue and note and display the data ordered by first appearance. For the dialogue, the speaker information may also be conveyed to the user.

FIG. 10C illustrates an exemplary table storing association between two data sources.

An exemplary embodiment of the systems and methods described herein include data storage of matched associations. Storage of matched associations can take many different forms. Exemplary alternatives are provided here as examples only. A basic example may be to store matched associations by sentence number. Each match association could be assigned a unique id per clinic visit. Those match associations that are higher-level, e.g. GROUP or REPEATS, can reference identification numbers of match associations. An example of this is shown in Tables 5-6.

TABLE 5
Example of match associations table for non-complex sets

| clinic_visit_id | setid | label | note_sentence | dialogue_sentence |
|---|---|---|---|---|
| 001 | S01 | statement | 0 | 0 |
| 001 | S01 | statement | 0 | 1 |
| 001 | S02 | qa | 10 | 30 |
| 002 | S01 | dictation | 0 | 1 |
| . . . | . . . | . . . | . . . | . . . |

TABLE 6
Example of matched associations table for complex sets

| clinic_visit_id | setid | label | Target_setid |
|---|---|---|---|
| 001 | S03 | repeat | S01 |
| 001 | S03 | repeat | S02 |
| . . . | . . . | . . . | . . . |

However, this method may become brittle if the sentence tokenization method of the document changes. For example, one sentence tokenization method may count a semicolon as a sentence-ender, while another may not. Another storage method could be to store associations related to notes according to text offsets and audio-related data as time-stamps.

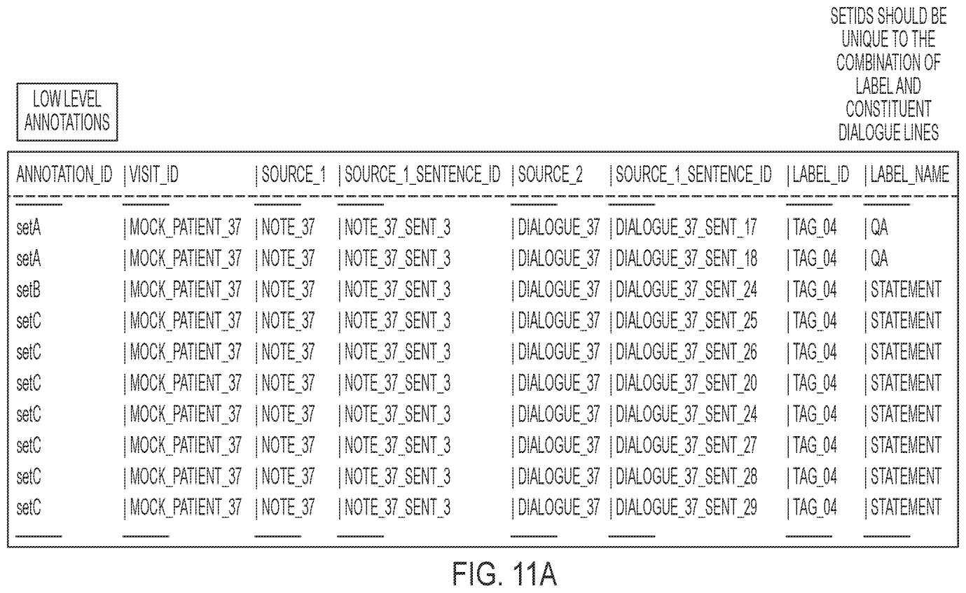

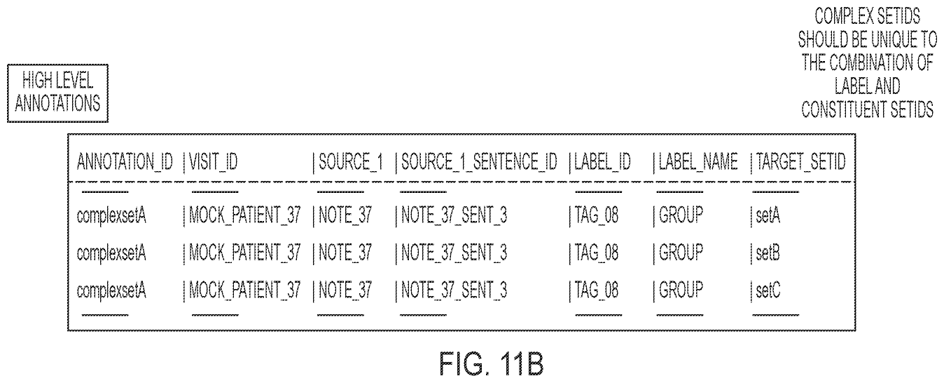

FIGS. 11A-11B illustrate exemplary tables and data sets according to embodiments described herein.

There are many variations that are encompassed by the instant disclosure to achieve the objectives described herein. For example, for the clinic visit dialogue, instead of having written text, embodiments may include data represented as speaker-tagged audio chunks. Thus, each audio chunk, through another type of interface can be attributed to a note sentence, by drag and drop assignment, for example. In an embodiment, exemplary associations may be made on different tokenized sizes. As described herein, a sentence is used as an exemplary data size. The sentence may be the smallest unit of match for a note. In exemplary embodiments, this smallest unit size may be larger for certain cases. For example, for parts of clinical notes for which data is structured, the unit size of the associated data may be different or larger. Examples include an automatically generated table of lab results or a grouped subsection of a template. Associations between the dialogue and the note may be based on location within the medium rather than on content.

### Generated Sentence Alignment

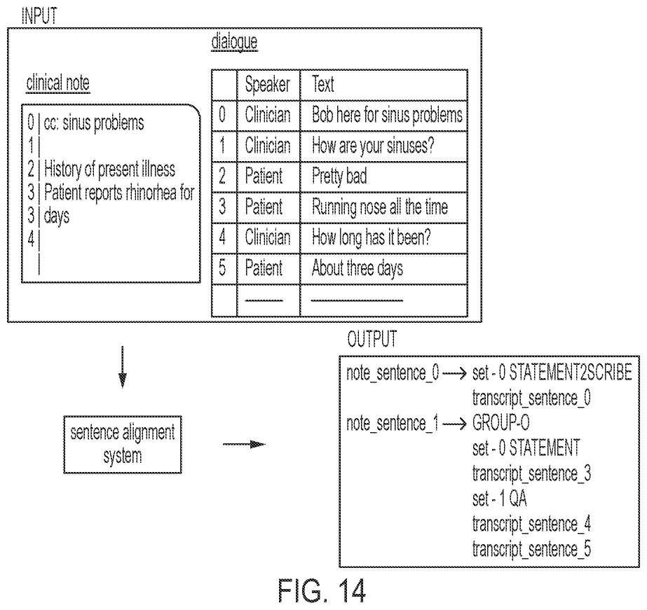

FIG. 14 illustrates an exemplary system input and output for the sentence alignment system. Paired source (a clinic visit dialogue) and target document (clinical note document) are inputs to the sentence alignment system and methods associated therewith. The result of the sentence alignment system is that each target document sentence is associated with 0 or more dialogue sentence sets.

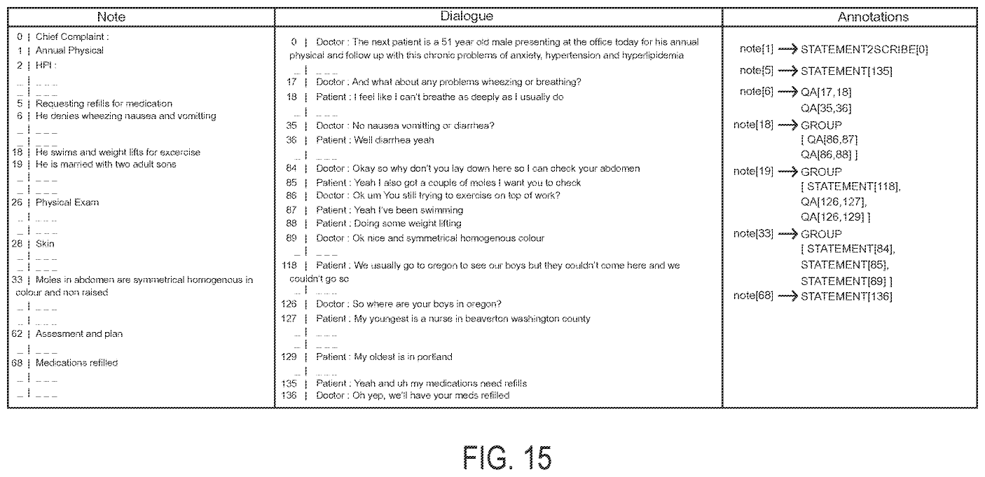

FIG. 15 illustrates an exemplary associated dialogue (MIDDLE) and clinical note (LEFT) shown along with their associated paired sentences organized into labelled sets (RIGHT).

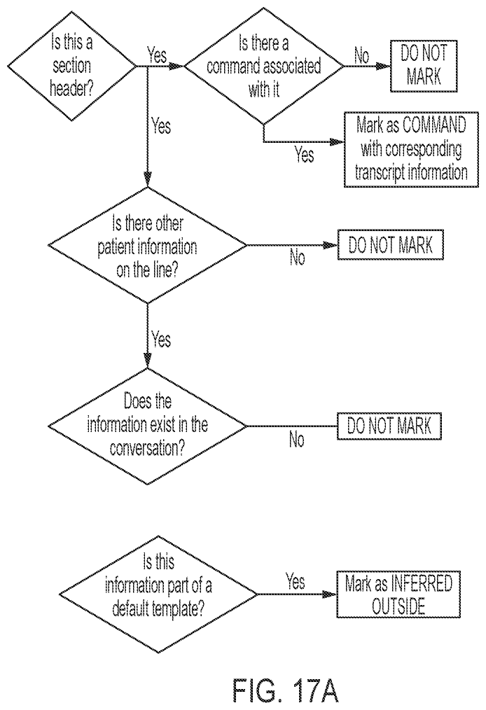

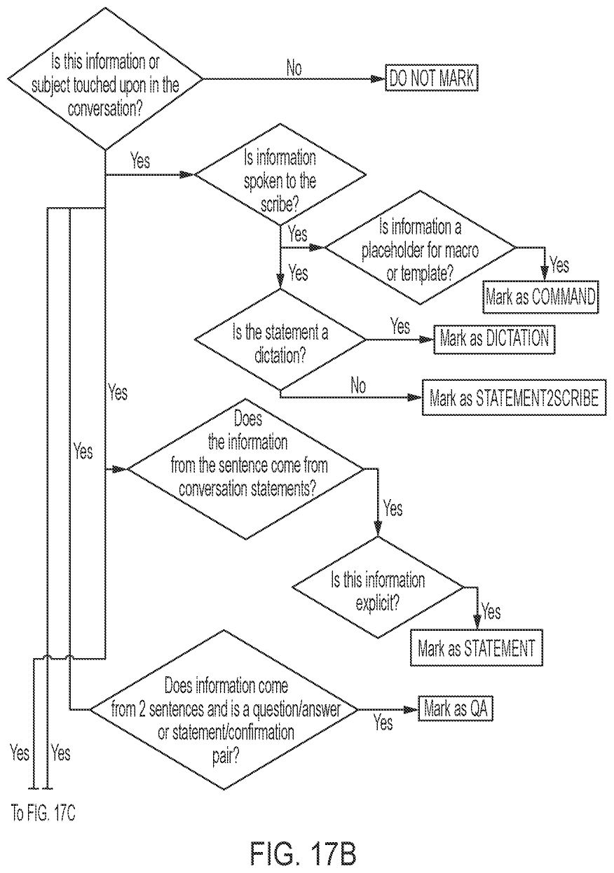

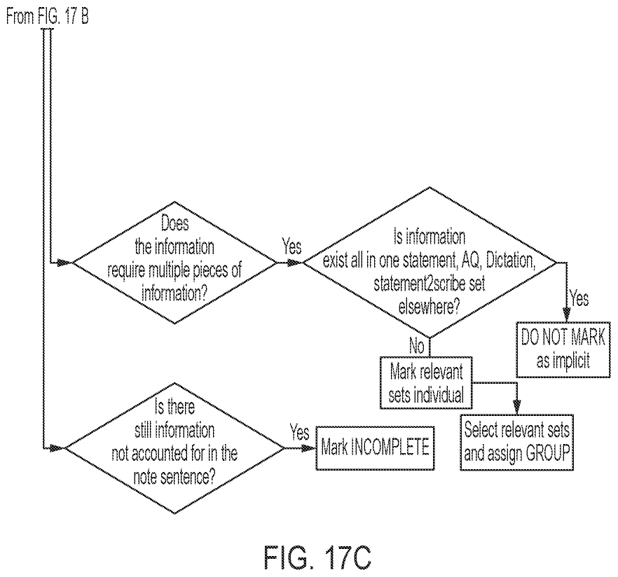

FIGS. 17A-17C illustrates an exemplary method for assigning tags according to embodiments described herein. For example, after having received or defined a sentence of the corpus, the system may analyse it to determine an associated tag to apply thereto. The system may analyse whether the sentence is a section header. If it is, the system may the analyse whether there is a command associated with it. If there is, then the sentence may be marked with a COMMAND tag, but if not, then the sentence may not be marked. The sentence may also be analyse to determine if other patient information is presented. If there is not, then no additional tag is marked. If additional information does exist, then the system may determine if the same information exists somewhere else. The system may prioritize one identify of the information over another. In this case, if the information occurs in the section header, it may not be marked, favouring or prioritizing the other location of the information. The system may then determine if the information in the sentence is part of a default template. If so, then the sentence or information may be tagged with INFERRED OUTSIDE.

Moving to FIG. 17B, the system may determine if the sentence or information in the sentence is part of the conversation. If not, then the sentence may not be marked, if it is, then the system may analyse the sentence further. For example, the system may determine whether the sentence is in a form directed at the system or scribe. If the information is an instruction for a macro or a template, the sentence may be marked as COMMAND. If the information is spoken to the system, but does not provide an instruction regarding a setting of the system, the system may determine that the statement is a dictation. If it is a dictation, then the sentence may be marked DICTATION. If it is not a dictation, then the system may mark the sentence as STATEMENT2SCRIBE. If the information is not spoken to the system or a scribe, the system may further assess the sentence to determine if the information from the sentence comes from conversation. If it does, and the information is explicit, the sentence may be marked as a statement. If the information comes from conversation, the system may also assess whether the information is composed over two or more sentences and/or in what format the information is provided. For example, if the information spans more than one sentence and there is a question and answer or statement and confirmation pair, then the sentences may be marked as QA. If the information includes multiple pieces of information and all of the information exists over the set of sentences, then each sentence may be marked individually with their associated tag and the group marked with the GROUP tag. The sentence may still be analysed to determine if there is any information remaining in the sentence not accounted for. If there is still information remaining that is not accounted for, then the sentence may be marked as INCOMPLETE.

Exemplary embodiments of the system may be tagged by a human scribe using, for example, the user interface as described herein. The system may also or in combination be automated, such that system modules may be configured to receive a corpus, receive and/or define a sentence within the corpus, analyse the sentence according to embodiments described herein, and assign tags and/or metadata as described herein.

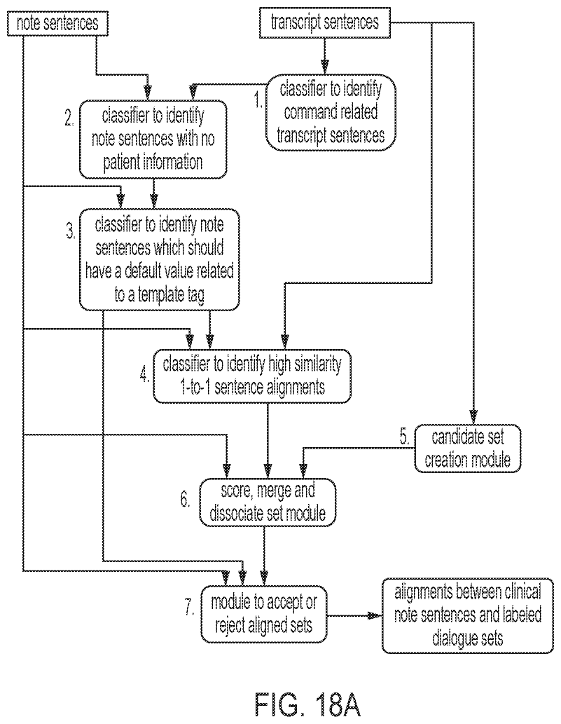

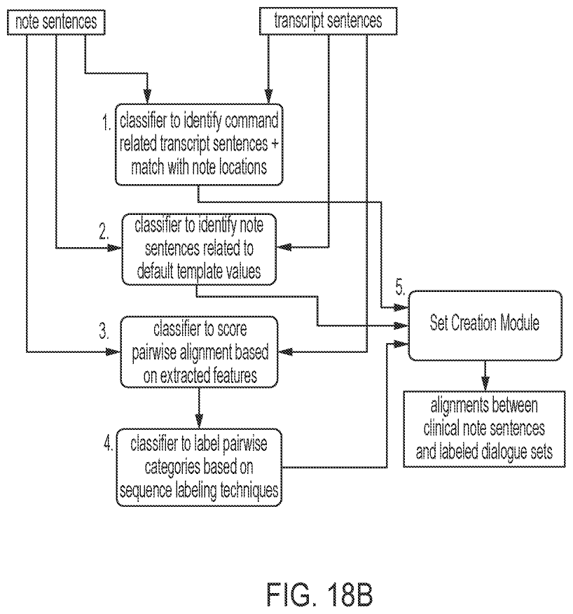

In an exemplary embodiment, the system and methods described herein may analyses the corpus and/or sentences within the corpus in sequential segments, simultaneously, or combinations thereof. For example, exemplary embodiments of the alignment task where the command-related tags, template-related tags, and remaining tags are treated and aligned separately. After the tags are assigned, the aligned sentences may be organized into labelled sets. FIGS. 18A-18B illustrates an exemplary system description according to embodiments described herein. The inputs to the system may include the paired clinical not and dialogue transcript from a clinical visit. The output may include an alignment between the note sentences and the labelled sets of transcript sentences.

Exemplary embodiments of the system described herein may include modules for performing the functions described herein. The modules may comprise machine readable instructions stored in a non-transitory machine readable medium, that when executed by a processor is configured to perform the functions described herein. The modules may be software, hardware, and combinations thereof. The modules described herein are representative only and does not necessitate separate codes. Instead, functions and modules may be combined, separated, duplicated, or recombined in any combination and stay within the scope of the instant description.

In an exemplary embodiment, the system may include a first classifier configured to identify command related transcript sentences. The first classifier may also or alternatively match the transcript sentence to the note location. In an exemplary embodiment the first classifier module is used to identify transcript sentences that are related to commands given by a clinician to a scribe or the system regarding changes to the clinical notes.

In an exemplary embodiment, the first classifier module may be used to identify when a clinician gives a verbal command that can result in alteration of the note structure and its corresponding note location for which the alteration may take place. For example, in a dialogue, a command given by the clinician could be to “insert a Flonase template” and the appropriate corresponding note location would be in the “plan” section of the clinical note.

Exemplary embodiments of the first classifier module may first identify transcript sentences which are commends. The first classifier module may then classify the appropriate note location for that command. This may be modelled as two classifications or may be modelled jointly. For example, to identify the required command transcript sentences, based on hand-features or neural network embeddings, transcript sentences may be classified as COMMAND or NOT-COMMAND. Given each command and a labelled note location, the next classification identifies a note section related to the command. The second task may also be modelled jointly with the first task such that along with COMMAND and NOT-COMMAND, the classifier would output a note location.

In an exemplary embodiment, the system may include a second classifier configured to identify note sentences related to default template values. The second classifier may be a module used to identify sentences with patient information, but may be included as a template default.

In an exemplary embodiment, the second classifier module may be used to identify portions of the clinical note which appear as part of default values from a predetermined template. For example, “Respiratory: regular rhythm” may appear in a template as the default value for a healthy individual. However, the clinician, whether or not they check this in the visit, may not verbalize this. Identifying these note sentences assists with correct disambiguation of which note sentences need to be matched with dialogue or not. This may be learned from training data, in which each instance is defined by a note sentence and a tag such as INFERRED-OUTSIDE or NOT-INFERRED-OUTSIDE. An example rule-based classifier can identify all saved templates' sentences and classify all matching candidate sentences as INFERRED-OUTSIDE.

In an exemplary embodiment, the system may include a third classifier configured to score pairwise alignments based on extracted features. The third classifier may be a module that scores alignment category matches for every note-to-transcript sentence pair given a set of features.

In an exemplary embodiment, the system may include a module for labelling pairwise categories based on sequence labelling techniques. The labelling module may take in pairwise scores from the third classifier, and finds the most likely sequence of labels for an entire transcript per each note line.

In an exemplary embodiment, the system includes a third classifier to score pairwise alignment based on extracted features and a labelling module to label pairwise categories based on sequence labelling techniques. These two modules can classify the sequence of transcript sentences in a visit, where each label is one of each associated label tags, such as STATEMENT, for each note sentence. For example, assuming for a visit, there are m note sentences and n transcript sentences. Then each note sentence can be associated with its own label sequence for n transcript sentences.

Each transcript sentence may be modelled by extracted hand-crafted features or by a deep learning embedding layer. The full sequence probability can then be calculated depending on the modelling assumptions. The simplest system may model each transcript sentence tag independently. Then maximizing the sequence could merely take the best classification for each sentence. However, to take previous or next labels into account, we can model using a hidden markov model, a maximum entropy markov model, a conditional random field (CRF), a neural network with a CRF layer, etc.

In an exemplary embodiment, the system may include a set creation module. The set creation module may be configured to receive the pairwise labels from the labelling module and creates sets therefrom.

In an exemplary set creation module, sets are created and organized with exemplary given available classifications, such as INFERRED-OUTSIDE, COMMAND, and other pairwise labels. An exemplary approach would be to create a set for every transcript sentence with a previously categorized label. However, another slightly more complex algorithm which groups together all consecutive transcript sentences with the same label as one set. Once base sets are created, higher order labels such as GROUP or INCOMPLETE tags can be added based on some classification criteria. Exemplary embodiments may include rules based assignment of tags, but assignment can also be posed as a machine learning tree creation task, similar to that of probabilistic content free grammar.

Exemplary embodiments may include systems and methods for defining alignments across dialogue text sentences and narrative text sentences. Exemplary embodiments may be used to organize associated dialog text into labelled sets. Exemplary embodiments may define the different subtasks and order of execution.

Exemplary embodiments include system and methods for automatically or computer controlled alignment of one corpus to another, where the first corpus may be a clinical dialog text made of one or more sentence structures and a second corpus, where the second corpus may be a clinical note text made up of one or more sentence structures. As used herein, a sentence structure of a corpus is any sub idea or sub text within the corpus. The sentence does not have to include or be isolated to a grammatically correct sentence structure. It may include one or more conventional sentence structures, or partial conventional sentence structures. In an exemplary embodiment a sentence structure within a corpus encompasses an idea that is relatable to another sentence structure of the other corpus. Exemplary embodiments of the aligned corpuses can be used for multiple purposes.

In an exemplary embodiment, the aligned corpuses may be used to create pre-annotations for an artificial intelligence assisted data labelling. Because automatic alignments may not be perfect, an automatic generated system may be used to augment human annotations by outputting suggested alignments for acceptance or modification by a human reviewer. The pre-annotations may assist with data entry and expedite the data processing.

In an exemplary embodiment, sentences marked as aligned by an automatic aligner can be used to train classifiers for transcript organization. For example, aligned dialogue sentences can be used as a first exposure to human classifiers for training for classifying relevant or irrelevant dialogue sentences. Sets pertaining to the same note sentence can be used as labels to train a classifier to group related sets.

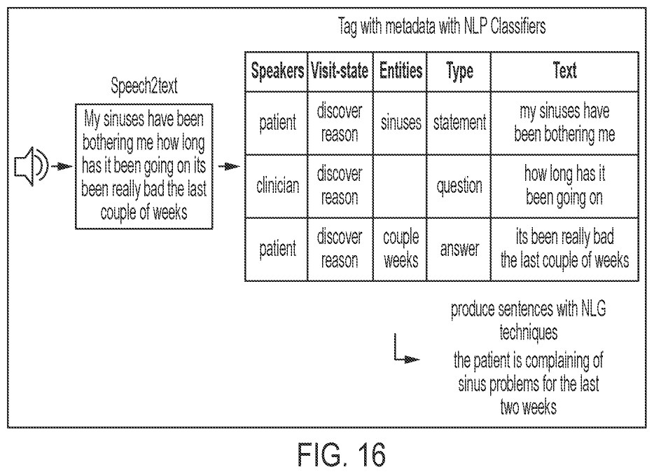

In an exemplary embodiment, aligned parallel data sets can be used to train a language generation system. For example, if a set of given paired alignments are provided, the corpus may be used to create a language generation system which would take in automatic speech generation data and output clinical note sentence suggestions, as shown in FIG. 16. As illustrated, exemplary methods include identifying and/or receiving a sentence from a corpus, wherein the corpus comprise text of a verbal dialog conversation between two individuals such as a patient and clinician. The system and method may be configured to extract information from the sentence, create metadata associated with the sentence, and/or apply one or more tags to the sentence. FIG. 16 illustrates an exemplary text extraction with associated analysis, metada, and/or tag assignment. As illustrated, metadata that can be identified and classified and/or stored with a corpus or sentence of a corpus; wherein the metadata may include an identity of the speaker (such as patient or clinician), a given visit state, specific key words (such as body parts, symptoms, duration, time, etc. Pre-defined or created in real time tags may be associated with the one or more metadata extractions, and/or sentences. For example, the tags used herein, such as to define a statement, question, answer, incomplete, etc.

In an exemplary embodiment, a system may include one or more of any of memory, processor, communication device, display, input device, output device, screen, keyboard, mouse, microphone, speaker, camera, video camera, network communication, sensor, temperature sensor, heart rate sensor, blood pressure sensor, blood oxygen sensor, blood glucose sensor, medical sensor, biological sensor, or other medical or electronic component. The system may, for example, include a microphone and/or video camera for receiving audio and/or video information. The system may, for example, include a communication for sending an receiving information from one system component part to another. The system may, for example, include a user interface for typing in transcription of the audio information. The system may, for example, include a digital sound receiver and transcription software for transcribing audio signals into written text information. The system may, for example, include a user input including mouse and keyboard for permitting a user to enter notes, impressions, comments, or other information. The system may, for example, include memory for storing the audio information, video or visual information, the transcribed or text information, or other information received/generated by the system. The system may, for example, include a user interface for displaying and receiving information from a user. The user interface may be configured to display one, two, or more information from different sources to the user. The user interface may be configured receive a user input. The system may be configured to link a first specific information of the one or more information from different sources to a second or more specific information of the one or more information from the different source. The system may be configured to also link a tag to the first specific information, the second or more specific information, and/or to the link of the first specific information to the second or more specific information. In an exemplary embodiment, the system may use a database to store data of, relating to, and/or identifying the first specific information, the second or more specific information, the link, the tag, or combinations thereof. In an exemplary embodiment, the system may be configured to assign and save a unique identification, such as an alpha-numeric data to a stored entry in the database. The unique identification may be used to separate, analyze, and/or identify a data entry and/or track relationships or information about one or more data entries. The system may also be configured to display information in one or more formats. For example, the system may have a first user interface comprising a display for receiving data and/or user input about one or more information sets as described herein. The system may have a second user interface comprising a display for providing information to the user about the received data and/or data sets as described herein.

FIG. 18A illustrates an exemplary embodiment of modules, in which the different modules are identified in the diagram as 1-7, identified below:

| | Module | Description |
|---|---|---|
| 1 | Classifier to identify | This module is used to identify transcript sentences that are related to |
| | transcript sentences related | commands given by a clinician to a scribe regarding changes to the |
| | to command tags | clinical note. |
| 2 | Classifier to identify | This module is used to identify sentences with no discernable patient |
| | sentences with no patient | information. This may include blank sentences, or section headers |
| | information | and/or may be tagged as associated with transcript lines related to |
| | | commands as outputted from the first module. This can be |
| | | probabilistic or rule based. |
| 3 | Classifier to identify | This module is used to identify sentences with patient information but |
| | template default value | may be included as a template default. |
| | sentences | For example: |
| | | Respiratory: regular rhythm |
| | | This may be learned from training data or use existing saved template |
| | | data for identifying matches. |
| 4 | Classifier to identify high | This module may be used to identify high similarity one-to-one |
| | similarity 1-1 alignments | alignments. |
| | | This may use different text similarity features, lexical, semantic, or |
| | | surface features for a number of different machine learning |
| | | classification techniques e.g., SVM, decision tree, etc. |
| 5 | Candidate set creation | Using a sliding window approach, sets of statements are organized as |
| | module | sets. Similarly question answer sets may be generated using proximal |
| | | sentences classified as questions and a following non-question |
| | | response. |
| 6 | Score, merge, dissociate | This may be a learned classifier used to create numerical ranking |
| | sets module | between a clinical note and candidate sets. |
| 7 | Classifier that accepts or | This classifier may accept or reject the aligned sets from module 6. |
| | rejects aligned sets | If an alignment set are rejected, then the note sentence may be |
| | | assigned to the default value which may be “no alignments” or to a |
| | | template-based tag value assigned in module 3. |

FIG. 18B illustrates an exemplary embodiment of modules, in which the different modules are identified in the diagram as 1-5. identified below:

| | Module | Description |
|---|---|---|
| 1 | Classifier to identify command | This module is used to identify transcript sentences that |
| | related transcript sentence and | are related to commands given by a clinician to a scribe |
| | match with note location | regarding changes to the clinical note. |
| 2 | Classifier to identify note | This module is used to identify sentences with patient |
| | sentences related to default | information but may be included as a template default. |
| | template values | |
| 3 | Classifier to score pairwise | This is a module that scores alignment category |
| | alignment based on extracted | matches for every note-transcript sentence pair given a |
| | features | set of features. |
| 4 | Classifier to label pairwise | This module takes in pairwise scores from Module 3, |
| | categories based on sequence | and finds the most likely sequence of labels for an |
| | labeling techniques | entire transcript per each note line. |
| 5 | Set creation module | This module receives the pairwise labels from Module |
| | | 4 and creates sets. |

Exemplary embodiments of the system described herein can be based in software and/or hardware. While some specific embodiments of the invention have been shown the invention is not to be limited to these embodiments. For example, most functions performed by electronic hardware components may be duplicated by software emulation. Thus, a software program written to accomplish those same functions may emulate the functionality of the hardware components in input-output circuitry. The invention is to be understood as not limited by the specific embodiments described herein, but only by scope of the appended claims.

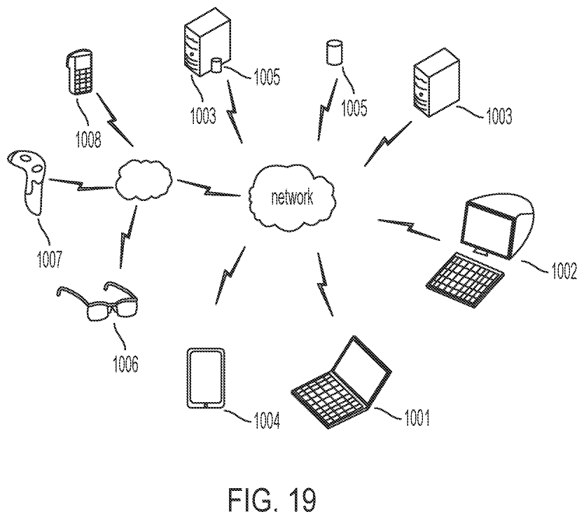

FIG. 19 illustrates an exemplary system configured to perform the functions described herein.

FIG. 19 illustrates an exemplary annotation system that can include embodiments described herein. Exemplary embodiments of the system and methods described herein may include a computer, computers, electronic device, or electronic devices. As used herein, the term computer(s) and/or electronic device(s) are intended to be broadly interpreted to include a variety of systems and devices including desktop computers 1002, laptop computers 1001, mainframe computers, servers 1003, set top boxes, digital versatile disc (DVD) players, mobile phone 1004, tablet, smart watch, smart displays, televisions, and the like. A computer can include, for example, processors, memory components for storing data (e.g., read only memory (ROM) and/or random access memory (RAM), other storage devices, various input/output communication devices and/or modules for network interface capabilities, etc. For example, the system may include a processing unit including a memory, a processor, an analog-to-digital converter (A/D), a plurality of software routines that may be stored as non-transitory, machine readable instruction on the memory and executed by the processor to perform the processes described herein. The processing unit may be based on a variety of commercially available platforms such as a personal computer, a workstation a laptop, a tablet, a mobile electronic device, or may be based on a custom platform that uses application-specific integrated circuits (ASICs) and other custom circuitry to carry out the processes described herein. Additionally, the processing unit may be coupled to one or more input/output (I/O) devices that enable a user to interface to the system. By way of example only, the processing unit may receive user inputs via a keyboard, touchscreen, mouse, scanner, button, or any other data input device and may provide graphical displays to the user via a display unit, which may be, for example, a conventional video monitor. The system may also include one or more large area networks, and/or local networks for communicating data from one or more different components of the system. The one or more electronic devices may therefore input a user interface for displaying information to a user and/or one or more input devices for receiving information from a user. The system may receive and/or display the information after communication to or from a remote server 1003 or database 1005.

In an exemplary embodiment, an exchange with a patient and clinician may be captured through one or more electronic device(s). For example, the exchange may be recorded including audio and visual. In an exemplary embodiment, an interface like an interactive glasses 1006 may be used that has a microphone for receiving audio input and a camera for receiving visual input. The patient exchange may be captured and recorded from the audio and/or image inputs. In an exemplary embodiment, the image input may permit a clinician to capture a picture, such as for visual record keeping of a symptom or condition of a patient. The image input may also provide a stream of images, such as a video exchange so that non-verbal interactions may be captured, observed, and/or recorded. Other electronic devices may be used to provide information to the system. For example, thermometer 1007 may provide a patient's temperature. Any other device 1008 may be used to capture information that can be digitized and stored or related to the patient information. This may be any other sensor information, video information, image information, audio information, etc. The captured information may comprise and define patient information. The patient information may be retrieved through any combination of interfaces, such as the described electronic device. However, the patient information may also be provided as preformed files, such as patient records, previously recorded or obtain information, such as prior exchanges with a clinician or captured data from one or more sensors for receiving and recording information about the patient. The exemplary patient information and sources of patient information are exemplary only and not intended to be limiting. The data input devices, including computers, recording devices, cameras, microphones, sensors, etc. are configured to transmit information over a network to other portions of the system described herein.

In an exemplary embodiment, the system may also include one or more computers. These computers may be in any form, such as desktop computer 1002, laptop computer 1001, or smart handheld device 1004, such as a phone or tablet. These computers are configured to provide a communication over a network to send and receive data from a remote server 1003. The remote server 1003 communicate with a database 1005.

In an exemplary embodiment, the system is configured as a computer 1003 and non-transitory computer readable storage medium storing a computer executable program for directing one or more processors to perform a method for configuring a pair of corpus according to embodiments described herein. The processors and/or memory may be local to a computer to the user. The system may include a downloadable software stored on a laptop computer 1001, desktop computer 1002, mobile device 1004. The software program may be stored in memory and when executed by a processor performs the steps described herein. The processors and/or memory may be remote, such as server 1003. The software program may be configured to display over a browser from a local computer, such as desktop computers 1002, laptop computers 1001, or mobile device 1004. Exemplary embodiments may include any combination of processors, memory, software programs and/or modules for performing the functions and methods described herein.

In an exemplary embodiment, the computer implemented method or software program may be configured to perform a method according to embodiments described herein. An exemplary method may include receiving a first corpus and a second corpus; matching a portion of the first corpus to a portion of the second corpus; and storing the matched association of the portion of the first corpus to the portion of the second corpus in a database.

Exemplary embodiments may include non-transitory computer readable storage medium storing a computer executable program for directing one or more processors to perform a method for configuring a pair of corpuses, wherein the pair of corpuses comprises a first corpus derived from a clinical exchange between a clinician and a patient and the second corpus is derived from the first corpus. The computer executable program may include a first classifier configured to identify commands related to sentences of the first corpus; a second classifier configured to identify portions of the second corpus that relate to default values from a predetermined template; a third classifier to label sentences of the first corpus; and a set creation module for creating sets between sentences of the first corpus and sentences of the second corpus.

In an exemplary embodiment, the first classifier may be configured to identify a verbal command given as an input that is not part of a dialog exchange between the clinician and the patient. The first classifier may be configured to determine an alteration in a structure of the second corpus related to the verbal command and determine a location of the alteration within the second corpus. The first classifier may be configured to tag each sentence of the first corpus as a command or not-command.

In an exemplary embodiment, the second classifier may comprise a rule-based classifier to identify all saved template sentences and identify matching candidate sentences from the second corpus with a given tag identifying the sentence as inferred from an outside source.

In an exemplary embodiment, the third classifier may be configured to provide a label for each pair of sentences, where each pair of sentences comprises a full pairing such that each sentence from the first corpus creates a matched pair with each sentence of the second corpus. For example, given n sentences from the first corpus and m sentences from the second corpus, each of the n sentences from the first corpus may be independently matched pair with each of the m sentences from the second corpus. For example, the first sentence from the first corpus may be matched with the first sentence of the second corpus, and separately the second sentence of the second corpus, and separately with the third sentence of the second corpus, and so on until the first sentence of the first corpus has been matched with the m sentence of the second corpus. The same sequential pairing can happen with the second sentence of the first corpus, and so on until the nth pairing of the first corpus with the m sentence of the second corpus. After the pairing, the complete pairing can be reduced by discarding at least one of the pair of sentences. The reduction can be to remove one or more sentence pairings from each of the sets of sentences pairings for each of the 1-n sentences from the first corpus. Therefore, the third classifier may be configured to maximize the generated labelled pair of sentences by discarding at least one of the pair of sentences for each of the sentences from the first corpus.

Exemplary embodiments described herein include a computer implemented method for marking a pair of corpuses for generating a machine learning library. The method may include providing a first corpus and a second corpus, matching a portion of the first corpus to a portion of the second corpus, and storing the matched association of the portion of the first corpus to the portion of the second corpus in a database. The method may also include receiving a tag related to the first corpus, the second corpus or the first and second corpus.

In an exemplary embodiment, the first corpus comprises a first textual representation of a dialog in a first language, and the second corpus comprises a second textual representation in the first language derived from the first corpus. The first textual representation of the dialog may be between a clinician and a patient, and the second textual representation comprises clinical notes. The first corpus and/or second corpus may be subdivided into a first plurality of sentences. The matching step of the method may then include one of the first plurality of sentences to one of the second plurality of sentences.

In an exemplary embodiment, the system and method may include displaying on an electronic display a user interface comprising a rendering of the first corpus and the second corpus. For example, the user interface may include a first section for displaying at least a portion of the first plurality of sentences and a second portion for displaying at least a portion of the second plurality of sentences. The matching may include receiving a user input through the user interface to select one of the first plurality of sentences and one of the second plurality of sentences.

In an exemplary embodiment, the first corpus may be a text representation of a verbal exchange and/or physical observations. In an exemplary embodiment, the first corpus may originate from an audio and/or video file of the exchange between the clinician and the patient. The first corpus may therefore originate from an audio file of a dialog that was converted to speaker separated text. Other text subdivisions may also be used.

In an exemplary embodiment, the system and/or method may automatically with the computer, identify sentences of the first corpus that are related to commands to the system regarding changes to a clinical note. The system and/or method may automatically with the computer, identify sentences of the second corpus that are related to a template default. The system and/or method may score an alignment match for every matching of a sentence of the first corpus to a sentence of the second corpus given a set of features to generate pairwise scores. Exemplary embodiments may use pairwise scores and determine a sequence of labels for an entire first corpus per each sentence of the second corpus creating pairwise labels. The system and/or method may use the pairwise labels to generate sets. The sets may be generated by creating a set for every sentence of the first corpus with a previously categorized label. The system and/or method may also apply higher order levels based on a classification criteria.

Exemplary embodiments, may create matched association of organized associations of sentences of the first corpus in labelled sets with sentences of the second corpus.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components and/or steps are described herein. Any combination of these components and/or steps may be used in any combination. For example, any component, feature, step, function, or part may be integrated, separated, sub-divided, removed, duplicated, added, moved, reordered, or used in any combination and remain within the scope of the present disclosure. Embodiments are exemplary only, and provide an illustrative combination of features, but are not limited thereto.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

What is claimed:

1. A computer implemented method for marking a pair of corpuses for generating a machine learning library, the method comprising:
providing a first corpus and a second corpus, wherein the first corpus is an audio file of a dialog which has been converted to a speaker separated text and the second corpus is a text;
matching a portion of the first corpus to a portion of the second corpus; and
storing the matched association of the portion of the first corpus to the portion of the second corpus in a database.

2. The method of claim 1, further comprising receiving a tag related to the first corpus, the second corpus or the first and second corpus.

3. The method of claim 2, wherein the first corpus comprises a first textual representation of a dialog in a first language, and the second corpus comprises a second textual representation in the first language derived from the first corpus.

4. The method of claim 3, wherein the first textual representation of the dialog is between a clinician and a patient, and the second textual representation comprises clinical notes.

5. The method of claim 1, wherein the first corpus is subdivided into a first plurality of sentences and the second corpus is subdivided into a second plurality of sentences, and the matching step comprises matching one of the first plurality of sentences to one of the second plurality of sentences.

6. The method of claim 5, further comprising displaying on an electronic display a user interface comprising a rendering of the first corpus and the second corpus.

7. The method of claim 6, wherein the matching comprises receiving a user input through the user interface to select the one of the first plurality of sentences and the one of the second plurality of sentences, the user interface comprising a first section for displaying at least apportion of the first plurality of sentences and a second portion for displaying at least a portion of the second plurality of sentences.

8. The method of claim 5, further comprising automatically with the computer identifying sentences of the first corpus that are related to commands to the system regarding changes to a clinical note.

9. The method of claim 8, further comprising automatically with the computer identifying sentences of the second corpus that are related to a template default.

10. The method of claim 9, further comprising scoring an alignment match for every matching of a sentence of the first corpus to a sentence of the second corpus given a set of features to generate pairwise scores.

11. The method of claim 10, further comprising using the pairwise scores and determining a sequence of labels for an entire first corpus per each sentence of the second corpus creating pairwise labels.

12. The method of claim 11, further comprising using the pairwise labels to generate sets.

13. The method of claim 12, wherein the sets are generated by creating a set for every sentence of the first corpus with a previously categorized label.

14. The method of claim 13, further comprising applying higher order levels based on a classification criteria.

15. The method of claim 5, wherein the matched association are organized associations of sentences of the first corpus in labeled sets with sentences of the second corpus.

16. A non-transitory computer readable storage medium storing a computer executable program for directing one or more processors to perform a method for configuring a pair of corpuses, the method including steps of:
receiving a first corpus and a second corpus wherein the first corpus is an audio file of a dialog which has been converted to a speaker separated text and the second corpus is a text;
matching a portion of the first corpus to a portion of the second corpus; and
storing the matched association of the portion of the first corpus to the portion of the second corpus in a database.

17. A non-transitory computer readable storage medium storing a computer executable program for directing one or more processors to perform a method for configuring a pair of corpuses, wherein the pair of corpuses comprises a first corpus derived from a clinical exchange between a clinician and a patient which has been converted to a speaker separated text and the second corpus is derived from the first corpus, the computer executable program comprising:
a first classifier configured to identify commands related to sentences of the first corpus;
a second classifier configured to identify portions of the second corpus that relate to default values from a predetermined template;
a third classifier to label sentences of the first corpus; and
a set creation module for creating sets between sentences of the first corpus and sentences of the second corpus.

18. The computer executable program of claim 17, wherein the first classifier is configured to identify a verbal command given as an input that is not part of a dialog exchange between the clinician and the patient.

19. The computer executable program of claim 18, wherein the first classifier is configured to determine an alteration in a structure of the second corpus related to the verbal command and determine a location of the alteration within the second corpus.

20. The computer executable program of claim 19, wherein the first classifier is configured to tag each sentence of the first corpus as a command or not-command.

21. The computer executable program of claim 20, wherein the second classifier may comprise a rule-based classifier to identify all saved template sentences and identify matching candidate sentences from the second corpus with a given tag identifying the sentence as inferred from an outside source.

22. The computer executable program of claim 21, wherein the third classifier is configured to provide a label for each pair of sentences, where each pair of sentences comprises a full pairing such that each sentence from the first corpus creates a matched pair with each sentence of the second corpus.

23. The computer executable program of claim 22, wherein the third classifier is configured to maximize the generated labeled pair of sentences by discarding at least one of the pair of sentences for each of the sentences from the first corpus.
